(19) United States
(12) Patent Application Publication
(10) Pub. No.: US 2009/0143777 A1
(43) Pub. Date: Jun. 4, 2009

(54) APPARATUS AND METHOD FOR TREATING TISSUE SUCH AS TUMOURS

(76) Inventors: Andrew Pacey, Herts (GB); Nagy Habib, London (GB)

Correspondence Address: OPPENHEIMER WOLFF & DONNELLY LLP, 45 SOUTH SEVENTH STREET, SUITE 3300, MINNEAPOLIS, MN, 55402, US

(21) Appl. No.: 12/297,844

(22) PCT Filed: May 23, 2007
(86) PCT No.: PCT/GB2007/001921

§ 371 Date: Feb. 3, 2009

(30) Foreign Application Priority Data
May 23, 2006 (GB) 0610637.1
Jan. 11, 2007 (GB) 0700560.6

(51) Int. Cl. A61B 18/10; A61B 18/04

(52) U.S. Cl. 606/27; 607/113; 607/99

(57) ABSTRACT

A catheter (4) may be introduced into the body of a patient to provide electromagnetic power, such as RF power, directly to a stent (2) to cause heating of surrounding tissue for ablation. The stent may have a conducting portion (14) and insulated portions (13). Struts (15) on the catheter may be deployed by a balloon (103) to contact the stent. The stent may have radial or sector segments (16) which may be individually powered for treating asymmetric tumours.

The present invention relates to a device and method for the treatment of tissue such as obstructive tumours surrounding or within lumens or vessels such as the oesophagus, trachea and bile duct or any other lumen which may be obstructable.

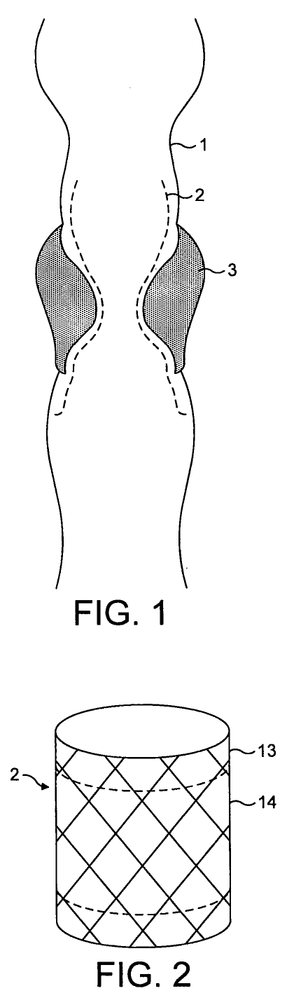

When tumours form around vessels, within vessels or within the wall of a vessel they can grow to surround the body of the vessel and cause obstruction of the lumen, which will have serious medical implications. A conventional method of treatment of such obstructions is the insertion into the vessel of a stent such as that shown in FIG. 1. This stent (2) is inserted into the lumen (1) to maintain patency of the blockage (3). Whilst in the short term this method keeps the lumen open, allowing matter to pass through the vessel normally, in the longer term this method has the drawback that the lumen may not return to its original dimensions due to the constraining effect of the tumour. In addition, the insertion of a stent into a vessel does not prevent tumour growth, therefore, hyperplasia can occur wherein the tissue re-grows inside the stent which will block the lumen further.

To overcome the problems associated with conventional stent methods, it is desirable to maintain patency of a blockage caused by a tumour and at the same time treat the tumour in order to restrict or reduce its size. U.S. Pat. No. 6,238,421 (Günther et al) discloses a system and method for heating cells surrounding metallic implants such as stents. An RF (radio frequency) electric signal is applied to an induction coil creating an alternating magnetic field inside the coil. When the portion of the being containing the metallic implant is positioned within the coil aperture, the magnetic field creates a heating effect on the metallic implant which in turn causes thermal damage to those cells surrounding the implant. The induction coil needs to be large enough to accommodate at least a portion of the person or other living being having a metallic implant. Another significant disadvantage of this method of heating cells surrounding stents is that the inductive heat is applied only to the metallic stent, and not directly into the tissue. Thus the tissue itself is heated only by thermal conduction from the stent. The thermal conduction is limited in its penetration of the surrounding tissue and is likely to be non-uniform as the temperature increase at the stent will fall away rapidly. However, the metallic implant will heat uniformly and will therefore uniformly damage the surrounding tissue, regardless of whether it comprises a tumour or healthy tissue. It is therefore desirable to have a compact device for generating localised heating of a tumour in a vessel. Furthermore, it is desirable to heat only tissue which comprises the tumour and not healthy tissue, the heating of which can lead to perforation of the vessel. Furthermore, it is desirable to deposit heat directly into the tissue without relying on heating by thermal conduction.

In US 2005/0125046, a stent may be heated from an externally applied alternating current field. Uniform heating will occur such that healthy tissue may be undesirably heated.

It is an object of the invention to alleviate at least to a certain extent the problems of the prior art.

The invention is set out in the claims. A compact and affordable method of applying a voltage, or other forms of power such as cyclic pressure power e.g. ultrasonic, directly to a stent may be provided. Furthermore, by provision of multiple struts connected to a catheter, selective heating of particular tissue areas can be obtained.

In particular, the invention enables power, such as radio frequency (RF) or other electromagnetic power or cyclic pressure power to be applied to a stent (or other implant) at regular intervals, for example weekly, in order to shrink a tumour whilst causing minimal damage to surrounding healthy tissue. Because a catheter can be in some preferred embodiments inserted into the stent via a body orifice, the invention is advantageous when treating, for example, elderly patients, while the alternative of performing surgery on the tumour would be a much riskier option. A direct physical connection to the stent/implant from outside the body allows good control of which part of the stent/implant is to be actuated.

Once a catheter in accordance with some embodiments has been inserted into the stent it is supplied with an RF voltage from the RF generator to which it is connected. Other forms of power, e.g. microwave or ultrasonic are also envisaged. The voltage applied and the duration and frequency of this application can be varied according to the nature of the tumour. Furthermore, according to one embodiment of the invention, individual struts of the catheter to which the RF voltage is applied can be separately deployed and can be supplied with varying levels of RF voltage dependent on the nature and shape of the tumour. The application of RF voltage to the stent causes the heating of the tissue surrounding the stent which causes desiccation and ablation of the tissue resulting in shrinkage. Use of microwave frequency is also envisaged.

Direct application of power e.g. by physically touching a stent, allows good control of which tissue near a stent is to be heated and does not require a patient to be accommodated within a large piece of apparatus as in the prior art. The device and method of the present invention allows the user to sufficiently treat tumours within vessels/lumens at regular (or other planned) intervals whilst causing minimal damage to healthy tissue and at the same time to prevent lumen obstruction which the presence of such tumours can cause.

Embodiments of the invention will now be described, by way of example, with reference to the drawings of which:

FIG. 1 shows a prior art stent in situ within a lumen;

FIG. 2 shows a front view of a stent in accordance with an embodiment of the present invention;

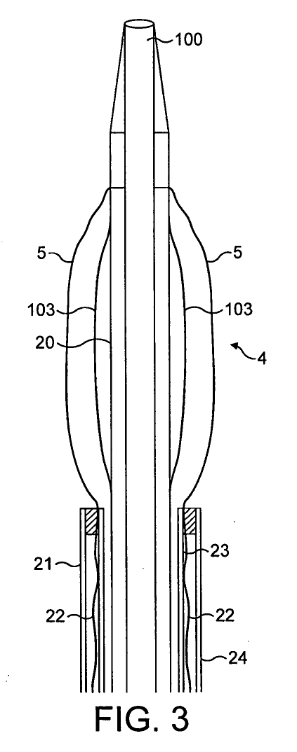

FIG. 3 shows a front view of a catheter according to a first embodiment of the present invention;

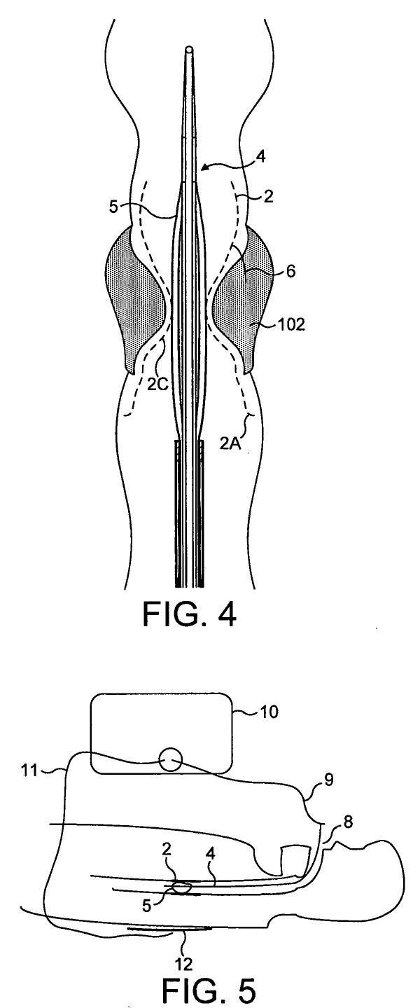

FIG. 4 shows a front view of the catheter of FIG. 3 and the stent in situ within a vessel;

FIG. 5 shows the electrical arrangement of the catheter, stent and a radio frequency generator;

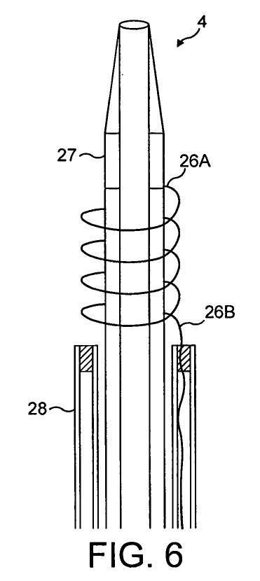

FIG. 6 shows a front view of the catheter according to a second embodiment of the present invention;

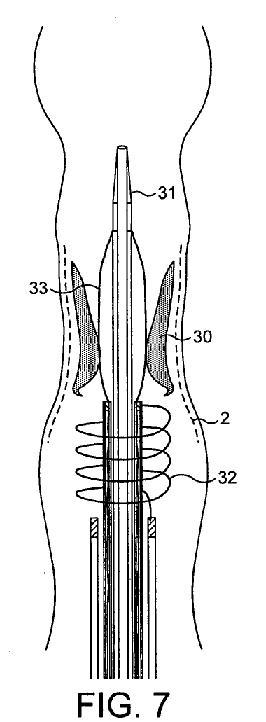

FIG. 7 shows a catheter according to a third embodiment of the invention and the stent in situ within a vessel;

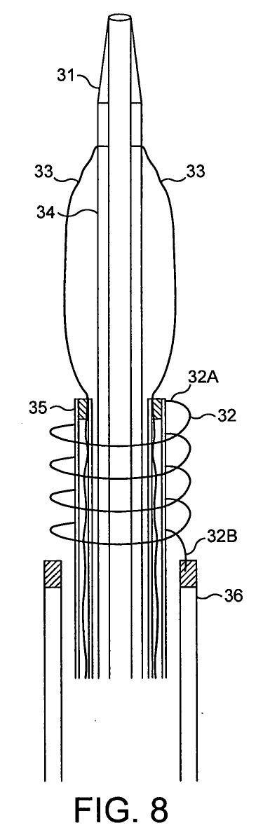

FIG. 8 shows a front view of the catheter of FIG. 7;

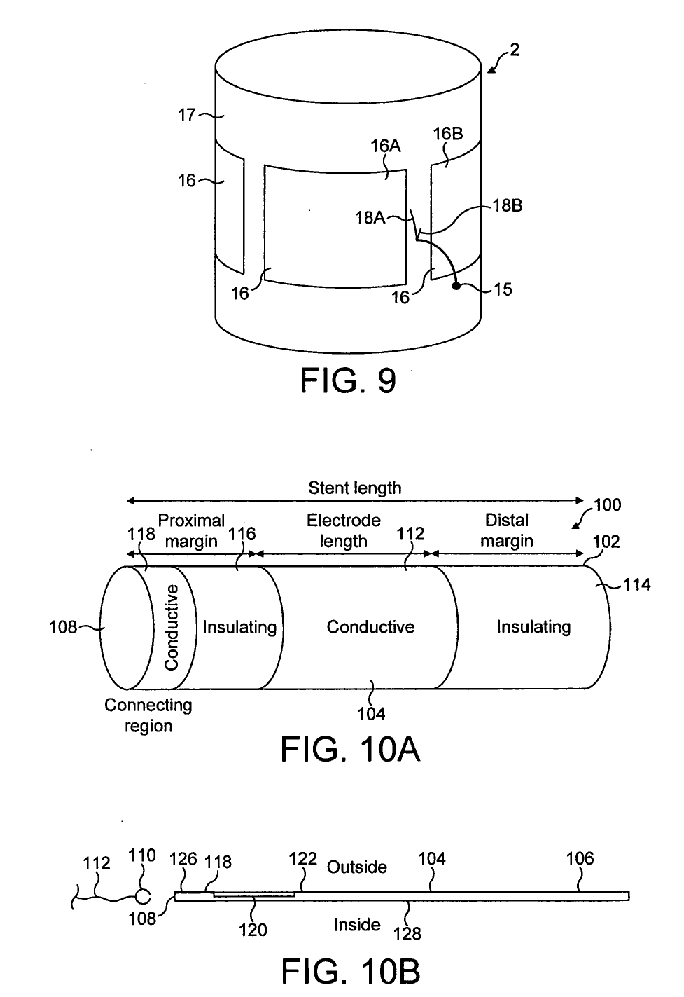

FIG. 9 shows a front view of an alternative embodiment of the stent;

FIGS. 10A and 10B show side and sectional views, respectively, of a further embodiment of the stent;

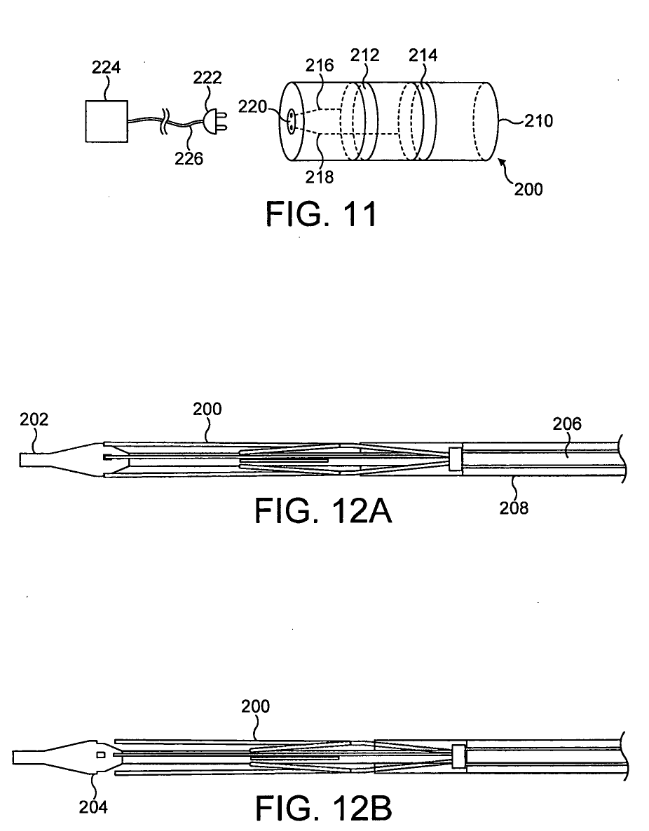

FIG. 11 shows an embodiment of a stent having plug/socket connection;

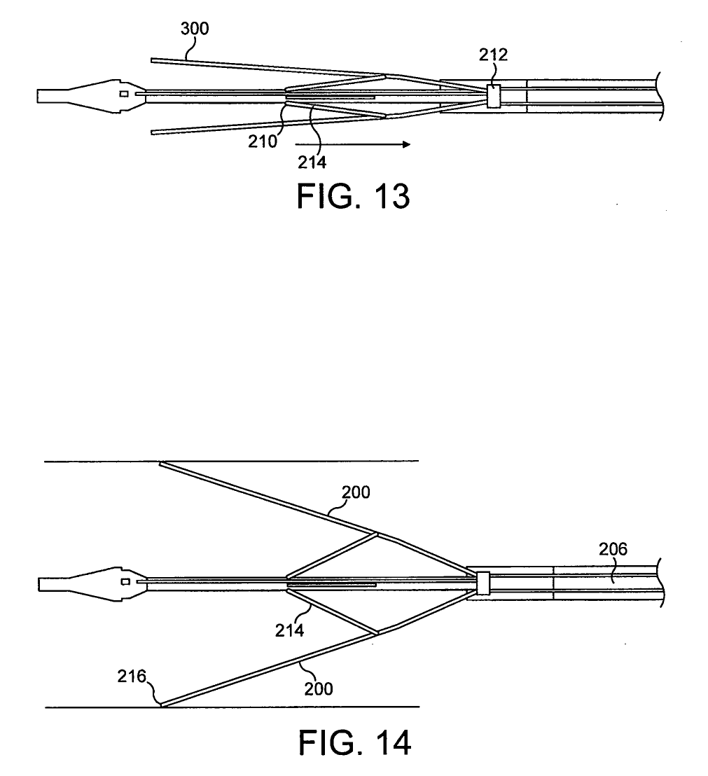

FIGS. 12A, 12B, 13 and 14 show a preferred embodiment of a device similar to the device of FIG. 2 in which needles or struts may be expanded to contact and supply power to a stent or the like;

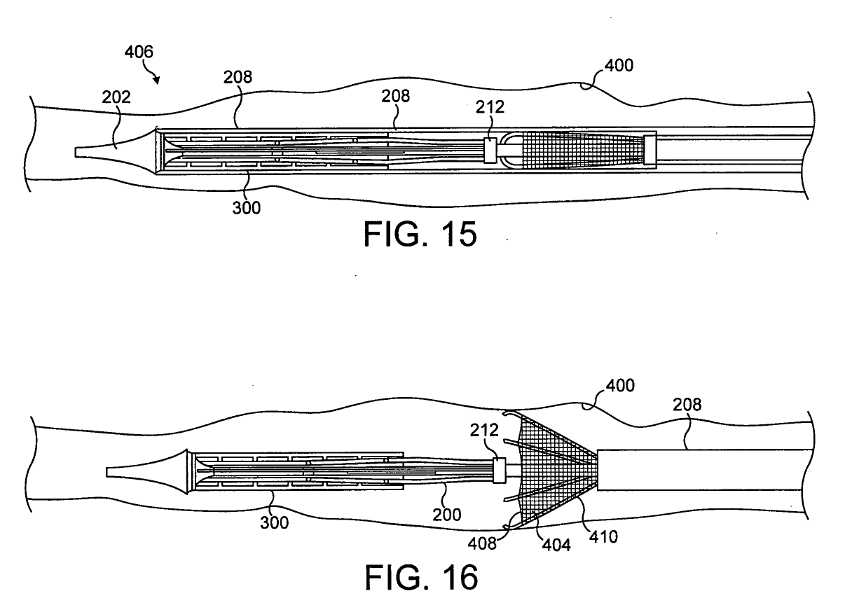

FIG. 15 shows a stent and deployment/powering device together with a stent in a collapsed configuration for percutaneous/endoscopic application;

FIG. 16 shows the device of FIG. 15 with a sleeve thereof retracted and a filter net thereof expanded;

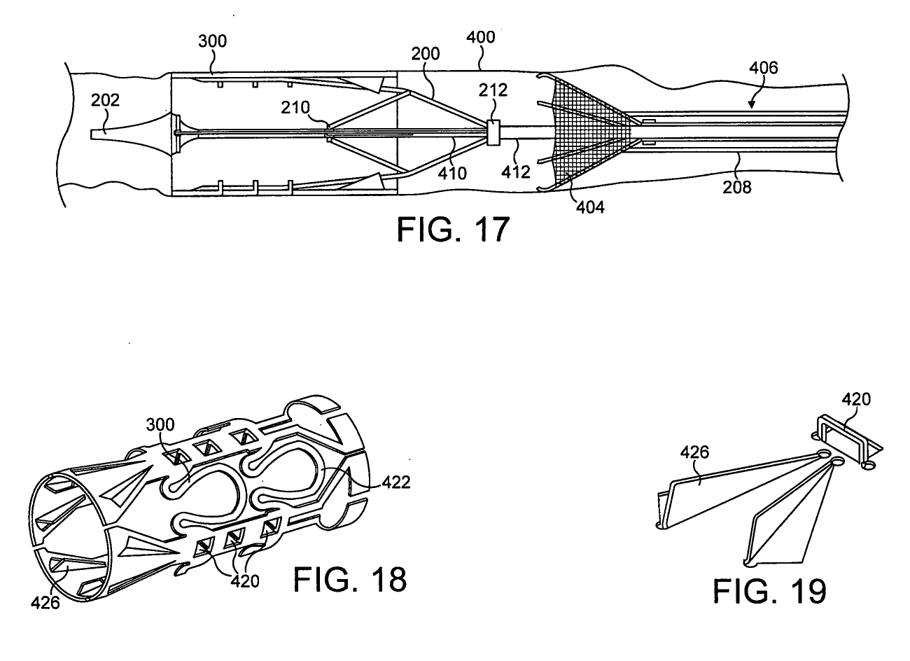

FIG. 17 shows the device of FIG. 15 and FIG. 16 with struts expanded so as to enlarge the stent;

FIG. 18 is an isometric view of the stent of FIGS. 15 to 17;

FIG. 19 is an enlarged view of alignment lugs and a loop located on the inside of the stent of FIG. 18;

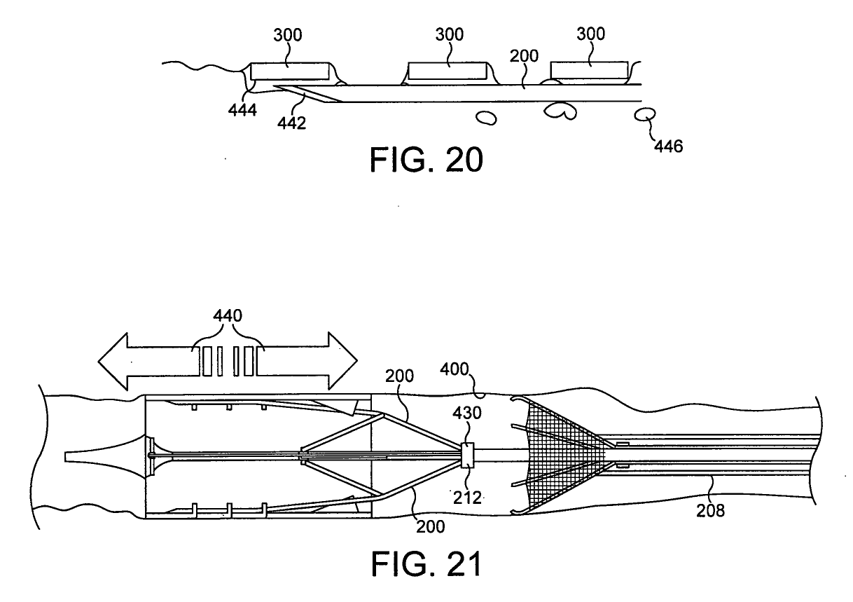

FIGS. 20 and 21 show how an ultrasonic vibration may be applied to the struts/needles of the device of FIGS. 15 to 17 so as to remove built-up material on the stent once it has been in position for sometime, so as to improve electrical contact between the struts and the stent for improved powering of the stent;

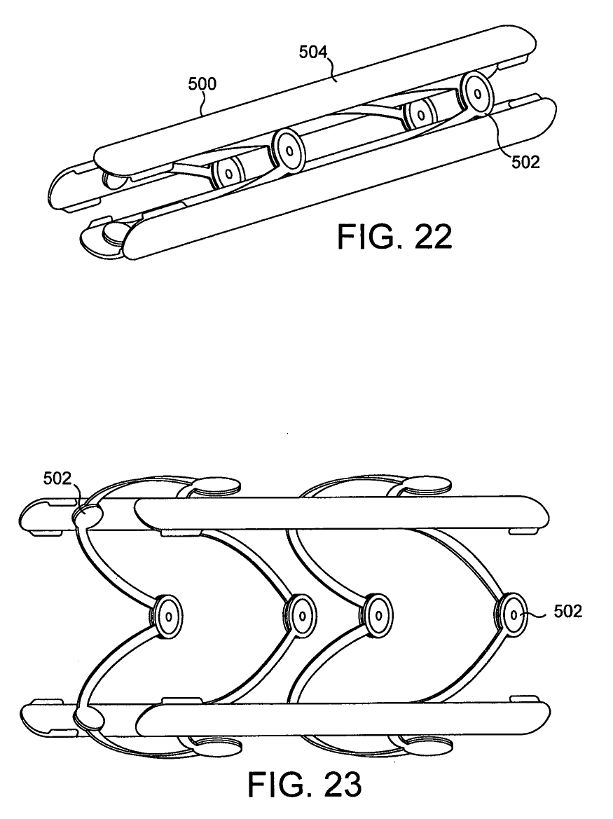

FIGS. 22 and 23 show a modified form of the stent of FIG. 18, in respective collapsed and enlarged configurations thereof;

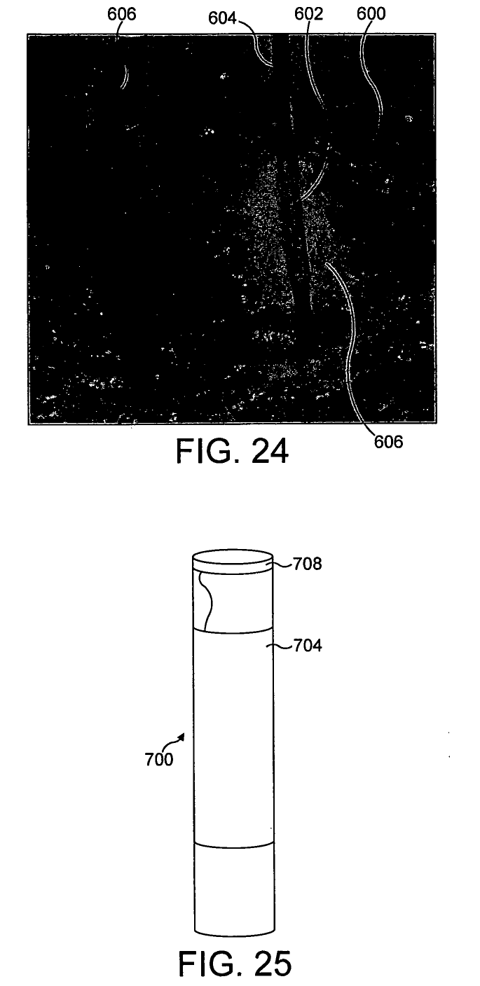

FIG. 24 shows an example of good remote direct heating of tissue near a stent using RF power in accordance with the concepts of the preferred embodiments; and

FIG. 25 shows a further example of a stent in accordance with an embodiment of the invention.

Referring to FIG. 2, the stent (2) of an embodiment of the present invention may be seen in detail. The stent (2) comprises a generally cylindrical mesh of metal layer with a hollow interior. According to the embodiment shown in FIG. 2, in order to prevent heating and ablation of normal tissue, the two end portions (13) of the stent (2) are insulated. The central portion (14) is un-insulated so that it will heat up adjacent tissue when a voltage is applied to the stent (2) as will be explained in more detail below.

FIG. 3 shows a catheter (4) (or introducer) used to apply RF voltage to the stent (2). The catheter (4) comprises an inner body (20) and an outer body assembly (21). A plurality of struts (5) are tethered at their distal ends to the inner body (20) and at their other ends are connected to the distal end of the outer body assembly (21). The outer body assembly (21) is arranged so that it can be slid longitudinally relative to the inner body (20) to deform the strut (5) size so that its shape can be changed from straight to a curved arc when deployed to contact the stent (2). The struts (5) are individually connected to connecting wires (22) which are contained in the space between the inner tube (23) and the outer tube (24) of the outer body assembly (21). As an alternative, the struts (5) could be connected to semi rigid wires (not shown) which are arranged to slide relative to the inner body (20) and the outer body assembly (21). In this arrangement the outer body assembly (21) is fixed with respect to the inner body (20).

As shown in FIGS. 4 and 5, in order to apply RF or other suitable voltage to the stent (2) in situ within a vessel, the catheter (4) is inserted into the hollow opening 2A of the stent (2) and, in operation, touches the stent (2) with struts (5). The catheter (4) can be inserted percutaneously or through a body orifice and connected via a wire (9) to one terminal of an RF generator (10). The other terminal of the generator is connected via a second wire (11) to a large patient electrode (12) of the type commonly used in medical RF applications. When a radio frequency voltage is applied to the stent (2) via the strut (5) and the wire (9), RF current will flow in the tissue surrounding the un-insulated section of the stent (2) and heat up this tissue to cause desiccation and ablation of the tissue, resulting in the shrinkage of tumour (102). The way in which the catheter (heating device) (14) touches the stent to apply power thereto provides direct application of power to the stent (2). Once the tumour (102) has shrunk, the obstruction of the lumen will have been alleviated. A balloon (103), which is shown partly inflated in FIG. 3 but omitted from other figures for clarity, and which is inflated in a manner similar to a conventional angioplasty balloon, can subsequently be further inflated to deploy the stent (2) further and increase the diameter of the lumen 2C.

In other embodiments, wires or contacts serving the same function as strut (5) may be located on outer surface of balloon (103).

The application of RF to the stent (2) via the catheter (4) can be repeated at regular intervals once the tumour (102) has shrunk after the last application. The frequency of the voltage and duration and frequency of the application can be varied according to the nature of the tumour. For example, the frequency applied may be in the range of 100 kH_{z} to 2 MHz and the voltage may be in the range of 10 Volts to 200 Volts. The frequency of application depends upon how the tumour/tissue shrinks, but may for example be weekly.

This method of regular application of radio frequency voltage in situ at the site of the tumour provides the means for treating the tumour whilst causing minimum damage to normal tissue.

As shown in FIG. 4, a probe (6) can be mounted on the outside of the stent (2) and embedded into the tumour (102). This probe (6) can monitor the temperature of the tumour (102) and will permit accurate control of the heating to avoid damage to normal tissues.

In addition, according to one embodiment of the invention, it is possible to deploy each individual strut (5) separately to touch and/or supply power to the stent (2). The struts in this case are loops on the catheter, as shown marked (33) in FIG. 8. In the case where a tumour is asymmetrical and therefore does not require the same degree of treatment throughout its circumference, the voltage applied to each strut (5) can be controlled according to the nature of the tumour tissue in the region which will be affected by the RF voltage supplied by that strut (5).

FIG. 6 shows an alternative embodiment of a catheter (4) used for connecting to the stent (2). In place of using struts (5), the connection is made by a helical arrangement of wire (26), tethered at one end 26A to inner body (27) and at the other end 26B to outer body assembly (28). The catheter (4) may be inserted into the stent (2) as described above. Once the catheter (4) has been inserted it may be deployed by rotating the outer body assembly (28) relative to the inner body (27). This will cause the helix to unwind and increase its diameter until such a point as it makes contact with the stent (2). Radio frequency voltage is then applied to the stent (2) via the helical arrangement of wire (26) which causes the same heating effect as described with respect to the first embodiment of the catheter (4).

FIG. 7 shows a situation where there is unwanted tissue (3) or hyperplasia inside the stent (2). The catheter (31) combines two contacting arrangements for making electrical connection to both the stent (2) and the unwanted tissue (30). In this example on the contacting arrangements is a helix (32) which makes contact with the stent (2) and the other contacting arrangement is in the form of deformable struts (33) which make contact with the tissue (30). Those contacting arrangements are connected to the two terminals of an RF generator of that RF current flows through the unwanted tissue (30) inside the stent (2) causing it to be ablated. If the unwanted tissue (30) is not circularly symmetric about the vessel, the ablation can be directed to different angular sections of the tissues by switching current to individual struts (33) or varying the level of current between individual struts (33).

FIG. 8 shows in more detail the combination catheter shown in FIG. 7. There are three concentric tubular assemblies. One end of each strut (33) is tethered to inner body (34). The other end is tethered to an immediate body (35). The struts (33) may be deployed by sliding the inner body (34) longitudinally relative to the intermediate body (35). One end (32A) of the helix (32) is also tethered to the intermediate body (35). The other end (32B) of the helix (32) is connected to the outer body (36). The helix is deployed by rotating outer body (36) relative to the other bodies. It will be appreciated that whilst FIG. 8 shows one example of a combination catheter other combinations of contacting arrangements are possible.

FIG. 9 shows a further embodiment of the present invention, wherein the stent (2) is divided into two or more conducting segments (16). The segments (16) can be either radial or a sector. The segments (16) are conductive and are separated from neighbouring segments (16) by an insulating portion (17). With the particular embodiment shown in FIG. 9, the catheter (not shown) used to heat the tissue surrounding the stent (2) is arranged to have a number of deformable connecting struts which align with the conducting segments (16). It is possible for the individual connecting struts to be separately connected to the RF generator, so that only one segment (16) is powered at a time. This permits localised heating around the stent (2). This will be beneficial when treating asymmetric tumours, for example those that do not completely surround the vessel.

In this embodiment one or more struts (6) are attached to the outside of the stent. The strut (6) as shown in FIG. 9 has a temperature probe (15) mounted at its distal end. The two terminals of the strut (6) are connected to different conducting segments (16A, 16B) at contacts (18A, 18B) which permit interrogation of the temperature probe (15) via the catheter (4). When the stent (2) is deployed, strut (6) will embed in the tissue outside the stent (2), and the temperature outside the stent (2) can be monitored. This temperature monitoring permits accurate control of the heating of the stent (2) which acts to minimize damage to normal tissue surrounding the tumour. The ability to heat different circumferential locations also avoids the need to heat and damage healthy tissues.

The stent (2) is constructed of metal wire, typically stainless steel or a shape memory alloy such as nitinol. Typically the metal wire takes the form of a mesh or grid although it will be understood that other possible configurations are possible. As already discussed, in order to prevent damage to normal tissue, it is desirable for at least a portion of the stent to be insulated. The main body of the catheter may be constructed from any appropriate material as will be obvious to the skilled person. The struts of the catheter are desirably made of a conductive and elastic wire such as stainless steel or a shape memory alloy such as nitinol. The insulation at the insulating portion (17) may be provided by a coated polymer such as parylene (Speciality Coatings Ltd).

FIGS. 10A and 10B show an embodiment of a bilary stent (100) comprising a continuous tube. The stent is not expandable and has a main body (102) which is typically a piece of plastic tubing. An electrode (104) has a cylindrical structure and is mounted on an outer surface (106) of the main body (102). This stent may be mounted so that part of it, namely a first connection end (108) thereof protrudes out of the bilary duct, into the duodenum. This means that an electrical connection can be made at the end (108) of the stent (100) using a clip (110) shown schematically in FIG. 10B which may be connected by an electrical wire (112) to a sort of electro magnetic power (not shown) similar to the generator (10) shown in FIG. 5. The clip (110) may be used since there is access to the outer surface (106) of the stent (100). The conductive electrode may be a metal coating on the tubular main body, deposited using sputtering or evaporation or other known metal coating techniques. Alternatively, the electrode may be a region of wound or woven wire located in the conductive electrode link section (112) of the stent (100). The electrode (104) may therefore be a segment of the tube with the remainder of the tube comprising an insulating portion (114) at a distal margin of the tube and an insulating portion (116) together with a conductive connection region (118) at a proximal margin portion of the tube. The connecting or contact region, which may not be in contact with tissue, may be formed in the way similar to the electrode (104). Connection is made from the contact region (118) to the electrode (104) via insulated wires (120) which may be imbedded in the wall (122) of the main body (102) of the stent (100), or via subsurface conducting tracks similar to those used in multilayer PCB's. Alternatively or in addition, the electrical contact portion (126) of the connection region (118) may be on the inside surface (128) of the stent (100) and, for example, may be connected to the outside electrode (104) using vias similar to those used in multilayer PCB's and in this case, with the contact region on the inside surface (128) electrical power may be provided using catheters similar to those shown with respect to the earlier embodiments described herein. One or more clips (110) may be attached to the conductor portion (126) of the electrode, whether it is on the outside surface (106) or inside surface (128) of the stent (100) by use of a connecting means delivered percutaneously, such as a catheter or endoscopic forceps or another endoscopic tool. The stent (100) may be operated in monopolar mode. It is envisage that a plurality of spaced conductive electrodes may be located in the conductive portion (112) of the stent (100) in some embodiments, with separate conductive power pass ways provided via connection region (118) to a generator for operation in bipolar mode.

FIG. 11 shows an embodiment of a stent (200) having a main tube (210) and at least two activatable electrodes (212, 214), each connected by an insulated electrical pathway (216, 218) to a distinct electrical connector 220. In this embodiment the connector (220) may comprise a socket (220) engageable by a plug (222) so as to connect stent (200) to power supply (224) located outside a body in which stent is located via wires (226), thus enabling operation in bipolar mode. Plug 222 is removeably attachable to socket (220) using endoscopic or percutaneous connector means. Similar plug/socket connectors may be used for stents operable at microwave frequencies, or at RF frequencies if desired.

Whereas the present invention has been described as using radio frequency power it will be appreciated that any appropriate electromagnetic power may be applied to the stent in order to achieve the desired heating result. For example, it is possible to activate the stent using microwave power.

Also envisaged is the use of other forms of power applied at the stent to cause tissue heating, such as physically vibrational or cyclic pressure power such as ultrasonic.

Before power is supplied to the stent it is desirable to obtain an accurate assessment of the tumour or area of tissue to be treated. This assessment may be obtained using external ultrasound equipment or by using endoscopic ultrasound scanning. In a further embodiment of the present invention, the catheter further comprises means for carrying out endoscopic ultrasound scanning of the tissue surrounding the stent before the power, e.g. electromagnetic power, is applied. This enables the surgeon or other user to determine the amount of energy which should be delivered through the stent to various areas of the tumour or other tissue.

In other embodiments it is envisaged that the stent may be controlled by an on-board chip or a chip located near the stent for switching in chosen conducting regions on the stent which will alter the regions around the stent which are heated up during treatment.

The invention as described herein has several advantages for both the user and the patient. The catheter provides energy to the stent in situ within the vessel wherein the tumour or other tissue is formed. Because the energy is supplied in situ and not from a source external to the body, this minimises potential damage to healthy tissue which does not need to be treated. Furthermore, the energy can be applied via the catheter at regular intervals in order to reduce tumour size without causing excess damage to the healthy tissue surrounding the tumour. The invention, in preferred embodiments, provides a method of selectively applying varying levels of electromagnetic energy to individual areas of tissue depending on their density and nature of tissue at that point.

Because the catheter can be inserted percutaneously or though a body orifice, this method of treatment is minimally invasive and therefore advantageous to the patient. The present invention can be used to reduce the size of the tumour within a vessel as an alternative to surgically removing the tumour. In addition, with the present invention there is no need for large and expensive equipment such as induction coils. This should allow the preferred embodiments to be easily obtainable for medical practitioners and therefore widely available to patients.

In the device of FIGS. 12A to 14, which is similar to the device of FIG. 2, the needles or struts (200) may be expanded to contact and supply power such as RF or microwave power to a conducting stent (300) or the like. As shown, a soft tip (202) guided by a guide wire is provided with a locking cone (204). The operator transforms the device through the steps shown in FIGS. 12A to 14 in order, then activates the stent, using electromagnetic power applied from outside the patient along inner shaft (206) and along conducting flexible nitinol arms/needles/struts (200) which act as electrodes which contact stent (300) to provide power thereto for tissue heating. Outer sleeve (208) may be slidable on shaft (206) for covering or exposing arms (200). During loading of the device endoscopically or percutaneously into the in situ stent (300), the arms are locked in position in the locking cone (204) as shown in FIG. 12A. The locking cone (204) is engaged to allow deployment of the arms to start. As shown in FIG. 12B, a pivot collar (210) is pulled back towards a fixed collar (212) rotating stainless steel tie bars (214) to force distal tips (216) of arms (200) against the stent (300). Once the stent has been activated by power, the configuration may be reversed through the steps to the FIG. 12A configuration from the FIG. 14 configuration, outer sleeve (208) may be slid forward over the arms (200) towards the locking cone (204), and the device may be removed from the patient.

FIG. 15 shows a modification of the device of FIG. 12A during deployment into a lumen (400) of a patient. Similar reference numerals denote similar parts to those in FIG. 12A. In total, four arms (200) are provided. In the embodiment of FIG. 15, the arms (200) are flexible and, initially, the stent (300) may be contained within the outer sleeve (208). The sleeve (208) may be drawn back to expose the stent (300) and arms (200) and expand a filter cage (404) as shown in FIG. 16, once the catheter/device (406) has been located using a guide wire and x-ray. The filter net (404) may be mesh, fiber or balloon sock with micro holes (408) on a self-expanding frame (410) which expands automatically once the outer sleeve (208) is withdrawn past it. As shown in FIG. 17, the stent may be deployed by pulling pivot collar (210) towards pivot collar (212) by pulling on shaft (410) inside shaft (412) remotely from outside the patient. The arms (200) are shown in FIG. 17 to be engaged through loops (420) of stent (300). Once stent (300) has been expanded from the contracted configuration shown in FIG. 18 by the arms (200) to expanded configuration shown in FIG. 17, the expansion being allowed by the resilient hoops (422) of stent (300), arms (200) may be slid axially out of hoops (420) and the catheter (406) may be removed, including the outer sleeve (208), arms (200) and tip (202), leaving the stent (300) in place in the lumen (400). After a periodic interval, the catheter may be reinserted into the stent (300) to the configuration shown in FIG. 17 and FIG. 21, the guides (426) serving to guide the arms (200) into the loops (420). By virtue of an ultrasonic piezo electric transducer (430) located inside pivot point (212), and powered by cables running along inside outer sleeve (208), arms (200) may be vibrated in the direction of the arrows (440) shown in FIG. 21 and sharpened tips (442) of arms (200) may engage or scrape against or near inner surfaces (444) of stent (300) to remove built-up material (446) which has grown inside the stent since its deployment, the material (446) then being caught by the filter (404). Therefore, this process of ultrasonic piezo electric cleaning may improve electrical contact between the stent (300) and the arms (200) enabling power to be efficiently and reliably applied to stent (300) in order to heat tissue in the region thereof for ablation treatment purposes. In an alternative embodiment, the arms (200) need not be inserted into the loops (420) in order to clean the stent (300) using ultrasound. The ultrasound may produce a rotational force on the arms (200) rather than the longitudinal force shown in FIG. 21.

FIGS. 22 and 23 show a modified version of the stent (300) shown in FIG. 18, the stent (500) of FIGS. 22 and 23 having hinges (502). The stent (500) may have side bars (504) which are steel or which are plastics, including conducting portions thereon for RF or other heating purposes. The stent (500) may be deployed using the framework system of arms (200) shown in FIGS. 17 to 21 or in other cases a balloon could be employed for such deployment.

In certain cases, in the embodiment of FIG. 21, the process of ultrasonic rubbing itself may be sufficient to cause local heating and ablation of tissue in the region of the stent (300). However, the ability to clean these stent struts is advantageous for RF heating since the rubbing allows clean surfaces to make contact.

The material (446) may for example include biofilm, mineral, tissue, and/or fatty deposit causing stent occlusion.

The stent (300) shown in FIG. 18 may be a silver-palladium stent and this is intended to have the advantage of reducing build-up of biofilm thereon.

In some applications, a filter (404) is not required for the catheter (406) and filter (404) may therefore be absent in some embodiments, for example, when the catheter is first used to place the stent in position in the lumen/vessel of the patient.

A prototype stent (700) to the design of FIG. 25 with a diameter of 4 mm with a cylindrical electrode (704) was inserted into ex-vivo porcine muscle tissue. The proximal connection ring (708) was connected to one polarity of a radiofrequency generator via a suitable clip. A ground pad consisting of a 10 cm square of conductive foil in close contact with one side of the tissue was connected to the other polarity. The temperature of the inside of the stent tube was monitored with a thermocouple.

W of RF power at a frequency of 450 KHz was applied to the stent electrode. The initial impedance was 44 ohms, and the initial temperature 30° C. After 3 minutes of heating the temperature was 98° C., and the impedance had decreased to 35 ohms. Following the heating, the stent was removed and the tissue sectioned as seen in FIG. 24. The region of tissue coagulation is clearly seen as a cylinder of diameter 14 mm, and length 25 mm.

In an embodiment, a nitinol spring or clip (not shown) is used to retain a stent on a loading device such as a catheter. As the stent is heated, the clip swings open to allow the removal of the catheter from the stent. Once the stent cools, the clip returns to a closed position. The clip allows the stent to be removed from the lumen by re-engagement with a removal catheter or allows re-heating of the stent by re-location with an electrode catheter. Furthermore, the nitinol clip could be used to lock a plastic tube stent, which could then be removed from the lumen independently from a metal electrode stent.

FIG. 24 shows the successful result of testing the remote heating of tissue (600) by direct remote application of EM power to a stent (602) by a power delivery device in accordance with the general concepts disclosed herein. After cutting open the tissue, constant tissue heating, which was desired in this particular case, was demonstrated in the region of the stent, as shown by heated region (606).

It is envisaged that the stent or stent powering device may have at least one magnetic contact or a contactable magnetisable so as to provide good electrical contact to the stent during EM powering thereof to heat tissue.

Various modifications may be made to the examples described herein without departing from the scope of the invention as defined by the accompanying claims as interpreted under patent law.

What is claimed is:

1. A device for the treatment of tumours or other tissue surrounding or within a lumen, the device comprising a stent, wherein the stent is arranged to heat tissue by the direct connection of power using percutaneous or endoscopic connecting means.

2. A device for the treatment of tumours or other tissue formed within a lumen, the device comprising a stent, wherein the stent is arranged to heat tissues by the direct application of power by a powering device.

3. A device as claimed in claim 1 wherein the stent is arranged to be positionable within the lumen at the site of a tumour or other tissue to be treated.

4. A device as claimed in any of claim 1 wherein the stent is arranged to heat up tissue on the application of power to cause ablation thereof.

5. A device as claimed in any of claim 1 wherein the stent comprises at least one of an insulating portion and a conductive portion.

6. A device as claimed in claim 5 which includes a said conductive portion for applying current to tissue and a connecting region for connection to a heating device for providing power to the stent.

7. A device as claimed in claim 6 in which the connecting region comprising a connection pad located at one end of the stent.

8. A device as claimed in claim 6 in which the connecting region comprises a connection pad located inside the stent, the stent having a tubular form.

9. A device as claimed in claim 6 in which the connecting region is located on an outer surface of the stent.

10. A device as claimed in claim 6 which the conductive portion is located on an outer surface of the stent.

11. A device as claimed in claim 6 in which the conductive portion is spaced from the connecting region by a said insulating portion, and in which at least one insulated current path passes from the connection region via the insulating portion to the conductive portion to provide power to the conductive portion.

12. A device as claimed in claim 5 in which the conductive portion comprises a metal coating.

13. A device as claimed in claim 12 in which the coating is formed on a main body of the stent which is formed of plastics material.

14. A device as claimed in claim 5 in which the conductive portion comprises a region of wound or woven wire.

15. A device as claimed in claim 14 in which the conductive portion is formed on a main body of the stent which is formed of plastics material.

16. A device as claimed in claim 1 wherein the stent has a main body formed of plastics material.

17. A device as claimed in claim 1 wherein the stent is substantially cylindrical.

18. A device as claimed in claim 17 in which the stent is formed as a tube.

19. A device as claimed in claim 1 wherein the stent is laterally deformable.

20. A device as claimed in claim 1 further comprising a balloon, wherein the balloon is arranged to inflate and cause lateral widening of the stent.

21. A device as claimed in claim 1 further comprising a temperature probe.

22. A stent for a lumen of a body, the stent having an outer surface and a heating portion, the heating portion having an electrode arranged to apply current to tissue, the electrode being operable over only a portion of the outer surface.

23. A stent for a lumen of a body, the stent having in one configuration thereof a generally circumferential periphery, and a heating portion, the heating portion of the stent being arranged to heat tissue in the region of a portion of the periphery.

24. A stent as claimed in claim 23 which includes a plurality of conducting elements separated by at least one insulating portion for selectively heating selected segments of the stent.

25. A stent for a lumen of a body, the stent having a distinct electrical connector part and an operative heating part electrically connected to the connector part, the electrical connector part being arranged for connection to a power source, and the operative heating part being arranged to heat tissue in the region thereof upon application of power to the connector part.

26. A device as claimed in any preceding claim which is adopted for bipolar operation.

27. A device as claimed in any preceding claim which includes an electrical connector part formed thereon.

28. A device as claimed in claim 27 in which the electrical connector part comprises a plug or a socket.

29. A powering device for the treatment of tumours or other tissue formed within a lumen comprising a percutaneous or endoscopic power delivery device (such as a catheter or endoscopic forceps) wherein the percutaneous or endoscopic power delivery device is arranged to apply power directly to a stent or implant within the lumen.

30. A device as claimed in claim 29 wherein the percutaneous or endoscopic power delivery device is arranged to apply power via a plurality of connecting struts, the struts preferably being deployable to move laterally relative to a longitudinal axis of the percutaneous or endoscopic power delivery device.

31. A device as claimed in claim 29 wherein the struts are deformable and may have either a straight or curved configuration.

32. A device as claimed in claim 29 wherein the struts are connected to a generator via a connecting wire.

33. A device as claimed in claim 29 wherein the struts are individually activatable with power.

34. A device as claimed in claim 29 wherein the percutaneous or endoscopic power delivery device is arranged to supply power via a helical wire.

35. A device as claimed in claim 34 wherein the width of the helical wire is variable, one end of the wire being rotatable relative to another end thereof to cause variation in width.

36. A device as claimed in claim 29 further comprising a temperature probe.

37. An apparatus for the treatment of tumours or other tissue surrounding or within a lumen comprising a powering device and a stent.

38. The apparatus as claimed in claim 37 wherein the powering device is arranged to be insertable into the stent.

39. A method of treatment of tumours or other tissue formed in or surrounding a lumen comprising:
(a) inserting a stent into a lumen in which there is a tumour or other area of tissue;
(b) applying power directly to the stent to heat the stent and ablate said tumour or other area of tissue.

40. A method as claimed in claim 39 which includes connecting a percutaneous or endoscopic connection device to the stent, and applying the power percutaneously or endoscopically.

41. A method as claimed in claim 39 which includes inserting a catheter into the stent and applying the power via the catheter.

42. A method of treatment as claimed in claim 39 in which the power is electromagnetic power at radio frequency.

43. A method as claimed in claim 39 wherein the power is microwave power.

44. A device as claimed in claim 1 which is arranged to heat tissue upon application of electromagnetic power.

45. A device as claimed in claim 1 which is arranged to heat tissue upon application of cyclic pressure power.

46. A device as claimed in claim 45 in which the cyclic pressure power comprising ultrasound power.

47. A device as claimed in claim 30 in which the struts comprise a framework on which the struts are pivotally connected together at one end thereof, with opposite free ends of the struts being laterally moveable to engage a stent and supply power thereto.

48. A device as claimed in claim 30 further including a vibration device for oscillating the struts to clean at least one electrical contact on a stent.

49. A device as claimed in claim 1 further including a magnetic component for securing electrical engagement between a stent and stent powering device.
